(19) United States
(12) Patent Application Publication
(10) Pub. No.: US 2007/0082061 A1
(43) Pub. Date: Apr. 12, 2007

(54) REDUCTION OF SALTINESS WITH SWEETENERS

(76) Inventors: Nelson Ayala, Lynchburg, VA (US); Cindy Minnix, Buchanan, VA (US); Michael Caswell, Lynchburg, VA (US)

Correspondence Address: EVAN LAW GROUP LLC, 600 WEST JACKSON BLVD., SUITE 625, CHICAGO, IL, 60661, US

(21) Appl. No.: 11/465,741

(22) Filed: Aug. 18, 2006

Related U.S. Application Data
(60) Provisional application No. 60/724,917, filed on Oct. 7, 2005.

(51) Int. Cl. A61K 33/42; A61K 31/7012; A61K 31/7024

(52) U.S. Cl. 424/601; 514/53

(57) ABSTRACT

The present invention provides compositions for bowel cleansing that have improved palatability through the inclusion of a sweetener, such as a chlorinated sucrose isomer. The invention also provides methods of reducing the saltiness of an orally consumed substance, including phosphate salt and PEG/salt bowel cleansers, through the use of a sweetener. Utilizing a sweetener including Sucralose to reduce the saltiness of a substance unexpectedly contradicts the conventional belief that sweeteners amplify saltiness.

### REFERENCE TO RELATED APPLICATIONS

This application claims the benefit of U.S. Provisional Application No. 60/724,917 entitled “Utilizing Sucralose to Reduce Saltiness” filed Oct. 7, 2005.

### BACKGROUND

Colonoscopy screening coupled with polyp removal (polypectomy) significantly reduces the incidence of colorectal carcinoma. Unfortunately, of the 147,500 new cases of colorectal carcinoma diagnosed in 2003, the American Cancer Society estimates that only 37% of these cases were diagnosed early enough for treatment to offer the best possible prognosis.

Colonoscopy screening should be repeated more frequently for subjects who have previously undergone a polypectomy due to their increased risk of recurrent polyp formation. However, in a follow-up phase of the National Polyp Study, at least 20% of subjects who had previously undergone polypectomies failed to return for their follow-up screening. In a more recent study, where 8,865 subjects who had previously undergone a polypectomy underwent a second colonoscopy screening, 2,704 (30.5%) were diagnosed with recurrent polyps. A statistical analysis based on the data from this report projected that 50% of subjects will have recurrent polyps within 7.6 years. Despite this level of risk, many subjects do not undergo additional screening.

Prior to colonoscopy, the bowel must be cleansed so the surgeon may see any polyps that exist on the interior wall of the colon. The bowel is the portion of the large intestine extending from the termination of the small intestine at the duodenum and extending to the rectum. Bowel cleansing generally entails the drinking of one or more laxative solutions. In addition to colonoscopy, bowel cleansers also may be used to cleanse the bowel before surgical and other endoscopic procedures.

Suitable laxative solutions for use as bowel cleansers include phosphate salt bowel cleansers or polyethylene glycol (PEG) combined with various salts. For example, phosphate salt bowel cleansers (monobasic and dibasic sodium phosphate), such as FLEET® PHOSPHO-SODA®, are very effective oral laxatives and are extensively used prior to colonoscopy, radiographic procedures, and surgery. For pre-colonoscopy use of PHOSPHO-SODA®, a split regimen is often preferred that includes one 45 mL dose given the evening before colonoscopy and a second 45 mL dose given at least three hours prior to the procedure on the following morning.

One of the main reasons subjects cite for avoiding colonoscopy re-screening is the unpleasant salty taste of bowel cleansing solutions. In fact, for phosphate salt bowel cleansers, the extremely salty taste of the solution is believed to be a cause of the nausea and vomiting that has been reported by from 15 to 51% of the subjects, depending on the study. Frequently, subjects cannot tolerate the ingestion of the complete initial dose of the preparation, which often prevents them from consuming more than a small portion of the second dose.

Thus, there is an ongoing need for bowel cleansers that taste less salty to the user and are thus more palatable. A better tasting pre-colonoscopy bowel cleanser could increase subject compliance with re-screening appointments and reduce the need for repeat procedures resulting from inadequate colon cleansing attributable to insufficient consumption of the cleanser solution. The materials and methods of the present invention provide bowel cleansers that are significantly less salty tasting and thus more palatable than conventional bowel cleansing solutions.

### SUMMARY

Colonoscopy screening coupled with polyp removal significantly reduces the incidence of colon cancer. Prior to colonoscopy, the colon must be cleansed so the surgeon may see any polyps that exist on the interior wall of the colon. Phosphate salt bowel cleansers, such as FLEET® PHOSPHO-SODA®, are very effective oral laxatives and are extensively used prior to colonoscopy. One of the main reasons subjects cite for avoiding colonoscopy re-screening is the unpleasant taste of the bowel cleansing solution.

The present invention makes use of the discovery that adding a sweetener, such as a chlorinated sucrose isomer, to a bowel cleansing solution, such as a phosphate salt bowel cleanser, significantly increases the palatability of the cleanser. The sweetener also may include Ace-K. The resultant sweetener/cleanser formulations may increase the subject's willingness to consume the cleansing solution, thus decreasing the repeat rate for initial colonoscopy procedures attributed to incomplete colon cleansing and increasing the subject's willingness to undergo follow-up procedures.

In a first aspect, the invention is a composition for bowel cleansing having a perceived saltiness equivalent to from 0.2 to 2.6% sodium chloride in water that includes from 0.01 to 0.1% of a sweetener selected from a chlorinated sucrose isomer, acesulfame potassium, saccharin, and mixtures thereof. In a related aspect, the perceived saltiness of the bowel cleanser and the sweetener amount may correspond to the relationship of FIG. 1 or Table 7.

In another aspect, the invention is a method of reducing the saltiness of an orally consumed substance having a perceived saltiness equivalent to from 0.2 to 2.6% sodium chloride in water where the substance is combined with from 0.01 to 0.1% of a sweetener selected from the group consisting of a chlorinated sucrose isomer, acesulfame potassium, saccharin, and mixtures thereof. In one aspect, the orally consumed substance may be bowel cleanser.

In another aspect, the invention is a method for improving the palatability of a bowel cleanser having a perceived saltiness equivalent to from 0.2 to 2.6% sodium chloride in water by combining the laxative with from 0.01 to 0.07% of a sweetener including Sucralose.

### BRIEF DESCRIPTION OF THE DRAWINGS

FIG. 1 shows the preferred Sucralose concentrations to reduce the saltiness of NaCI/water solutions of varying saltiness.

FIG. 2 plots the Likert preferability scores for multiple sweetener concentrations in a phosphate salt bowel cleanser.

### DETAILED DESCRIPTION

The present invention makes use of the discovery that adding a sweetener, such as one including a chlorinated sucrose isomer such as Sucralose, to a salty liquid, such as a bowel cleanser, significantly increases the palatability of the liquid by a reduction in perceived saltiness. In addition to a chlorinated sucrose, the sweetener also may include acesulfame potassium (Ace-K). Furthermore, depending on the saltiness of the liquid, a preferable sweetener concentration may be selected. Stable flavorings also may be added to the bowel cleanser to increase palatability. The resulting sweetener/salty liquid formulations may improve subject compliance for both primary screening of asymptomatic colorectal carcinoma, and for return surveillance in those subjects who may benefit from more frequent colonoscopy.

Phosphate salt bowel cleansers, such as commercially available FLEET® PHOSPHO-SODA® (C.B. Fleet Company, Inc., Lynchburg, Va.) taste extremely salty. Phosphate salt bowel cleansers include monobasic sodium phosphate (sodium dihydrogen phosphate, monohydrate) (NaH₂PO₄H₂O) and dibasic sodium phosphate (disodium hydrogen phosphate, heptahydrate) (Na₂HPO₄7H₂O) as active ingredients in water. Phosphate salt bowel cleansers have a pH from about 4.4 to about 5.2 and may be produced in multiple ways, such as by combining phosphoric acid with dibasic sodium phosphate or with caustic soda. Bowel cleansers of this type are very stable, thus having a long shelf-life, and are considered to work in a mild and very effective manner.

In one aspect, the phosphate salt bowel cleanser includes from 0.05 to 1.5 gram/mL of monobasic sodium phosphate and from 0.02 to 0.6 gram/mL of dibasic sodium phosphate. In another aspect, the phosphate salt bowel cleanser includes from 0.25 to 1 or from 0.4 to 1 gram/mL of monobasic sodium phosphate and from 0.1 to 0.4 or from 0.13 to 0.25 gram/mL of dibasic sodium phosphate. At present, an especially preferred phosphate salt bowel cleanser includes about 0.48 g/mL of monobasic sodium phosphate and about 0.18 g/mL of dibasic sodium phosphate. Phosphate salt bowel cleansers that include one phosphate salt, such as dibasic sodium phosphate, also may be used.

PEG based bowel cleansers, such as commercially available NuLYTELY® and GoLYTELY® from Braintree Laboratories, Inc., Braintree, Mass., also taste salty. While the active ingredient, PEG, lacks taste, the substantial amounts of salt, impart a salty taste to the bowel cleanser. Percent (%) compositions are expressed on a weight/weight (w/w) basis in the specification and appended claims, unless stated otherwise.

Although many sweeteners and flavorings exist, at least three significant factors must be considered when selecting sweeteners and/or flavorings to increase the palatability, thus reducing the perceived saltiness, of bowel cleansers. These factors are the ability of the sweetener to reduce saltiness, a lack of digestible sugars, and stability in the bowel cleanser solution.

Sweeteners and/or flavorings for use in bowel cleansers preferably exclude natural sugars that may be digested in the colon to form hydrogen gas, which may ignite during polypectomy. Furthermore, phosphate salts, for example, decompose most commonly available sweeteners and flavorings. As this decomposition of the sweeteners and/or flavorings proceeds, any palatability benefit gained from the sweetener and/or flavoring may vanish.

While it is possible to add the sweetener and/or flavoring a short time before consumption of a phosphate salt bowel cleanser and retain at least a portion of the palatability benefits, one goal of the present bowel cleansing compositions is to provide stable liquids having increased palatability. Thus, numerous sweeteners and flavorings were tested for stability in phosphate salt bowel cleanser. Of the sweeteners tested, chlorinated sucrose isomers, such as Sucralose, Ace-K, and Saccharin were found to have acceptable stability in solution.

Chlorinated sucrose is a no-calorie sweetener made by replacing three of the hydroxy groups (OH) of the sugar molecule with chlorine (Cl). The chlorine atoms are tightly bound to the sugar molecule, thus making it exceptionally stable. This stability is believed to prevent the body from digesting the molecule, allowing the chlorinated sugar molecules to pass through the body unchanged. The chlorination process may create multiple isomers of the sugar, depending on the reaction conditions and other variables. Sucralose is the common name for one of the isomers resulting from the chlorination process.

At present, Sucralose is a preferred chlorinated sucrose isomer for use in bowel cleansers. Sucralose is considered to be about 600 times sweeter than sucrose and to have a medium intensity of sweetness coupled with a relatively long-lasting sweetness in the mouth. While not wishing to be bound by any particular theory, it is believed that the stability provided by substituting the hydroxyl groups with chlorine atoms prevents the phosphate salts present in phosphate salt bowel cleansers from degrading chlorinated sucrose isomers.

Ace-K is a no-calorie sweetener made from the potassium salt of acetoacetic acid. Ace-K is very stable and not metabolized or stored in the body, thus passing through the body unchanged. Ace-K is considered to be about 200 times sweeter than sucrose and to have a high intensity and shorter lasting sweetness in the mouth. It also is considered to have a more “sugar-like” taste than other no-calorie sweeteners. The lack of hydroxyl groups may contribute to its stability in the phosphate salt bowel cleansers.

Saccharin is the oldest no-calorie sweetener and has been used to sweeten foods and beverages for almost 100 years. It is highly stable and is not digested, thus passing through the body without providing any calories. Saccharin is considered to be about 200 times sweeter than sugar and to have a slightly bitter aftertaste.

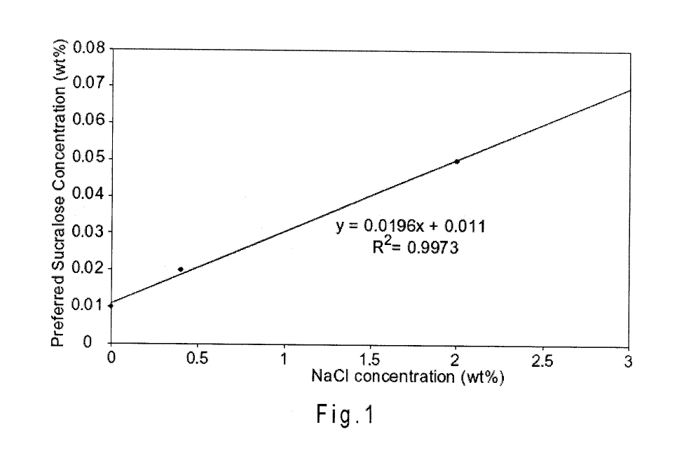

For liquids having a saltiness equivalent to NaCI/water solutions ranging from 0% NaCI to 2.4% NaCI, from 0.01 to 0.06% of a chlorinated sucrose isomer, such as Sucralose, is more preferred to decrease the saltiness of the liquid. Similar amounts of Ace-K and 5:1 Ace-K/Sucralose combination also are preferred. A 0.01 to 0.1% concentration of Saccharin also may be used. Thus, once baseline saltiness is determined for a specific liquid in relation to a NaCI/water solution, the preferred concentration of chlorinated sucrose isomer to reduce saltiness may be selected from FIG. 1.

For phosphate salt bowel cleansers, such as diluted FLEET® PHOSPHO-SODA®, from 0.01 to 0.2%, from 0.03 to 0.1%, or from 0.04 to 0.08% sweetener are preferred. For PEG based bowel cleansers, such as NuLytely® or GoLytely®, from 0.01 to 0.2%, from 0.01 to 0.08, or from 0.02 to 0.04% sweetener is preferred, with about 0.025% of the sweetener especially preferred at present for the NuLytely® cleanser. In one aspect, the sweetener includes one or more chlorinated sucrose isomers, with the Sucralose isomer being more preferred. The sweetener also may include Ace-K in combination with the chlorinated sucrose isomers. In one aspect, a ratio of about five parts Ace-K to one part Sucralose is preferred.

In addition to one or more sweeteners, many natural and/or artificial flavorings also were tested for palatability and stability in the phosphate salt bowel cleanser. Of the flavorings tested, ginger ale, such as Ginger Ale FAET253, mangosteen, such as Mangosteen FAES387, and cola, such as Cola FAES389, were found to have acceptable stability.

In one aspect the bowel cleanser includes from 0.3 to 2.3% flavoring. In a second aspect, bowel cleanser includes from 0.8 to 1.8% flavoring. In a third aspect, the bowel cleanser includes from 1 to 1.6% flavoring. At present, a phosphate salt bowel cleansers including about 1.3% of Cola WONF FAES389, Ginger Ale FAET253, or Mangosteen FAES387 is especially preferred. In PEG based bowel cleansers, these and other flavorings may be used.

By orally administering the sweetened or sweetened and flavored bowel cleansers of the present invention to a subject, the bowel may be cleansed. Generally, phosphate salt based cleansers are administered so that from 0.4 to 0.85 grams of monobasic sodium phosphate and from 0.1 to 0.5 grams of dibasic sodium phosphate per kilogram of body weight are consumed. A first aliquot of the cleanser may be administered to the subject about 14 hours prior to the colonoscopy. This initial dose may be followed by a second aliquot of the cleanser administered about 3 hours prior to the colonoscopy. The first dose may include an amount of phosphate salt bowel cleanser equivalent to 45 ml of FLEET® PHOSPHO-SODA® and the second dose may include an amount of phosphate salt bowel cleanser equivalent to 45 ml or 30 ml of FLEET® PHOSPHO-SODA®. The subject should consume large amounts of liquids, 3 to 4 Liters for example, in addition to the cleanser to maintain adequate hydration. These additional liquids may include aqueous solutions that include electrolytes, such as GATORADE® and other oral re-hydration beverages.

Generally, PEG based bowel cleansers are prepared by combining a dry PEG/salt combination with about 4 Liters of water. This solution is then consumed in 8 ounce portions every 10 minutes for nearly 3 hours. Due to the unpleasant taste of the solution and the large water volume, patients often do not consume the complete amount in the 4 hour maximum time period. The large volume of water may result in over-hydration and bloating. Furthermore, the colon begins to empty within about 30 minutes of consumption of the first 8 ounce portion.

In the examples below, it was unexpectedly discovered that subjects indicate a higher palatability for salty liquids that include a sweetener, in comparison to unsweetened salty liquids. Because palatability may be considered the inverse of saltiness, the addition of the sweetener was found to reduce the perceived saltiness of the bowel cleanser. The preferred concentration of a sweetener useful to provide the desired reduction in saltiness was also determined for solutions of varying saltiness. In this manner, a correlation was determined for the preferred concentration of a sweetener to combine with bowel cleansers of varying perceived saltiness.

It also was unexpectedly discovered that subjects significantly preferred phosphate salt bowel cleansers combined with a sweetener that included Sucralose in comparison to sweeteners that included Ace-K or Saccharin alone. Furthermore, it was determined that an approximate five to one mixture of Ace-K and Sucralose may be used as an acceptable substitute for Sucralose. The data below demonstrated that Sucralose and Sucralose containing sweeteners were effective at reducing saltiness and improving the palatability of bowel cleansers.

### EXAMPLES

### Example 1

Sucrose, Saccharin, Sucralose, Aspartame, Ace-K, Thaumatin (Talin), Neohesperidine Dihydrochalcone (NHDC), and Trehalose were tested for stability in FLEET® PHOSPHO-SODA®. Of these sweeteners, Sucralose, Ace-K, and Saccharin were found to have acceptable stability in the laxative. While the other sweeteners retained their effectiveness when mixed, their effectiveness diminished over time.

### Example 2

Phosphate salt bowel cleansers were prepared for preference comparisons. An exemplary composition was prepared as shown in the table below. The percentages are on a weight/weight (w/w) basis.

TABLE 1

| Ingredient | Amount (%) |
|---|---|
| Purified Water | 55.7 |
| Flavor - Cola WONF FAES389 | 1.3 |
| Sweetener | various |
| Phosphoric Acid (75%) | 16.6 |
| Dibasic Sodium Phosphate | 25.4 |
| Glycerin | 0.8 |
| Sodium Benzoate | 0.03 |

### Example 3

To determine whether test subjects preferred the taste of salty liquids with or without a sweetener, a hedonistic type preference test with 40 test subjects was conducted. Each subject was asked to rate two salty liquids on a scale of 1 to 9 with 1 being highly preferred and 9 being least preferred. Two percent NaCl/water solutions were prepared that included 1.29% ginger ale flavor.

The first NaCl/water solution (T in Table 2 below) included no sweetener, while the second NaCl/water solution (G in Table 2 below) included 0.13% Sucralose. Similarly, unsweetened (J in Table 2 below) and sweetened (K in Table 2 below) phosphate salt bowel cleansers were prepared. In comparison to the phosphate salt bowel cleanser of Example 2, these cleansers included 1.3% of ginger ale flavor instead of Cola.

TABLE 2

Salty Liquid

| Test Subject | T | G | J | K |
|---|---|---|---|---|
| 1 | 5 | 7 | | |
| 2 | | | 7 | 2 |
| 3 | 7 | 5 | | |
| 4 | | | 9 | 3 |
| 5 | 7.5 | 4.5 | | |
| 6 | | | 1 | 8 |
| 7 | | | 8.5 | 4 |
| 8 | 7 | 5 | | |
| 9 | | | 8.5 | 4 |
| 10 | 7 | 3 | | |
| 11 | | | 9 | 6 |
| 12 | | | 8 | 6 |
| 13 | 9 | 3 | | |
| 14 | | | 7 | 7 |
| 15 | 2.5 | 8.5 | | |
| 16 | | | 8.5 | 1.5 |
| 17 | 3.5 | 2.5 | | |
| 18 | | | 3 | 2 |
| 19 | 8.5 | 8.5 | | |
| 20 | | | 8 | 7 |
| 21 | 9 | 1 | | |
| 22 | | | 3.5 | 8.5 |
| 23 | 5.5 | 5.5 | | |
| 24 | | | 7.5 | 6.5 |
| 25 | 8.5 | 1 | | |
| 26 | | | 8.5 | 6.5 |
| 27 | 8 | 5 | | |
| 28 | | | 7 | 3 |
| 29 | 6 | 3 | | |
| 30 | | | 7.5 | 7.5 |
| 31 | 8 | 5.5 | | |
| 32 | | | 9 | 7 |
| 33 | 6.5 | 9 | | |
| 34 | | | 5 | 3 |
| 35 | 5.5 | 2.5 | | |
| 36 | 4.5 | 3.5 | | |
| 37 | | | 8.5 | 1.5 |
| 38 | 7 | 6.5 | | |
| 39 | | | 8.5 | 5 |
| 40 | 7 | 4 | | |
| Average | 6.6 | 4.7 | 7.1 | 4.9 |
| Standard Deviation | 1.8 | 2.3 | 2.2 | 2.3 |

As can be seen from the data in Table 2, the test subjects preferred sweetened salty liquids G and K over their unsweetened counterparts by approximately 47% for the two percent salt solution and by approximately 45% for the phosphate salt bowel cleanser. Thus, the unpleasant saltiness of an orally consumed liquid was reduced with Sucralose.

### Example 4

Before a preferred sweetener for use in salty liquids could be determined, the amount of each sweetener required to provide an equivalent sweetness was determined. Multiple taste tests were performed comparing various concentrations of Sucralose, Ace-K, the 5:1 Ace-K/Sucralose blend, Saccharin, and sucrose in a salty liquid to determine an equivalent saltiness. These tests established that 0.05% Sucralose, 0.05% Ace-K, 0.05% Ace-K/Sucralose, 0.083% Saccharin, and 15% sucrose provide an equivalent sweetness. While the remaining Examples rely on 0.05% sweetener concentrations, these may be converted to Saccharin concentrations using the 0.05/0.083 ratio.

### Example 5

To determine which sweetener was preferred to increase the palatability and thus decrease the saltiness of bowel cleansers, forty test subjects were asked to rate which of five cleansing solutions were most preferred on a scale of 1 to 4. Each solution was prepared by diluting 60 mL of the phosphate salt bowel cleanser from Example 2 in 355 mL of water and using either 0.05% Ace-K, 0.05% Sucralose, 0.08% saccharin, 0.05% of a 5:1 Ace-K/Sucralose blend, or 15% sucrose as the sweetener.

When the preference data was averaged, the average values obtained were 3.3 for sucrose, 3.2 for Sucralose, 2.8 for Ace-K/Sucralose, 2.5 for saccharin, and 2.45 for Ace-K. Thus, sucrose was most preferred followed by Sucralose, Ace-K/Sucralose, saccharin, and Ace-K. Surprisingly, Sucralose performed almost as well as sucrose. The Ace-K/Sucralose blend was superior to saccharin or Ace-K alone, but was not as preferred as Sucralose. Thus, while Sucralose alone was more preferred, the Sucralose/Ace-K sweetening system provides an alternative.

### Example 6

A comparison between Saccharin/Ace-K and Sucralose/Ace-K was performed to determine which of these two sweetener systems most improved the palatability of a salty bowel cleanser. Table 3, below, provides the mean acceptability ratings for 19 test subjects who tasted 5:1 Saccharin/Ace-K and 5:1 Sucralose/Ace-K sweetener systems in 60 mL of the phosphate salt bowel cleanser from Example 2 in 355 mL of water.

TABLE 3

| | Concentration | | Standard |
|---|---|---|---|
| Sweetening System | % (w/w) | Mean* | Deviation |
| Sucralose/Ace-K | 0.13 | 3.11 | 1.05 |
| Sucralose/Ace-K | 0.26 | 3.32 | 1.00 |
| Sucralose/Ace-K | 0.39 | 3.47 | 1.02 |
| Saccharin/Ace-K | 0.38 | 2.24 | 1.16 |

*Rankings were on a Likert scale from 1 to 5, with the following descriptors: 1 = unbearable; 2 = less preferred; 3 = ok; 4 = more preferred; 5 = great. Thus, the higher the value, the more preferred was the sweetening system.

The data confirm a substantial preference for Sucralose over Saccharin (up to about 55% at the same 0.38% concentration), with a slight increase in preference for higher Sucralose concentrations. While the linear trend of increasing palatability with increasing Sucralose concentration may not be significant (p=0.2593), the sweetening system that included Saccharin was judged significantly less palatable than any of the Sucralose systems (p =0.001 6).

While there were deviations in the mean acceptability ratings provided by the 19 testers, when averaged across the four sweetening systems, the deviations were not significantly different from the “noise” or error within ratings across the testers and sweetening systems (p=0.1175). Mean acceptability ratings for the testers were as low as 2.00 and as high as 4.25, illustrating the difference among people in the palatability of the phosphate salt bowel cleanser, regardless of the sweetening system.

### Example 7

To determine a baseline saltiness “taste” for bowel cleansers, such as 60 mL of the phosphate salt bowel cleanser from Example 2 in 355 mL of water, and the PEG cleansers, multiple test subjects rated the saltiness of 1, 1.5, and 2% solutions of sodium chloride (NaCI) in water. Of these NaCI solutions, it was discovered that a 2% solution of NaCI in water most closely approximated the saltiness of the phosphate salt bowel cleanser. Similarly, it was determined that a 0.4% solution of NaCI in water most closely approximated the saltiness of a PEG bowel cleanser including from about 420 to 240 g (10 to 6%) of PEG and from about 18 to 38 g (0.4 to 1%) of salt in water.

### Example 8

To establish the reliability of the NaCI to bowel cleanser comparison, test subjects were asked to taste sweetened and unsweetened NaCI solutions and sweetened and unsweetened phosphate salt bowel cleansers. In this comparison of the sweetened and unsweetened NaCI solutions, out of 19 test subjects, 17 preferred the 2% NaCI solution that included 0.13% Sucralose. Furthermore, in the preference comparison of the sweetened and unsweetened phosphate salt bowel cleanser, 18 out of 20 test subjects preferred the phosphate salt bowel cleanser including 0.13% Sucralose. Therefore, the palatability increase provided by the Sucralose was similarly observed for the 2% NaCI/water solution and the phosphate salt bowel cleanser.

### Example 9

To determine the preferred concentration of Sucralose to reduce the saltiness of bowel cleansers having different perceived saltiness, varying concentrations of Sucralose were tasted in water, a 0.4% NaCI water solution, and a 2% NaCI water solution. For each solution, approximately 40 testers were asked to rate four different concentrations of Sucralose. Tables 4, 5, and 6, below, present the average test data for the water, 0.4% NaCI water solution, and 2% NaCI water solution, respectively.

TABLE 4

| | (1-Dislike, | (1 -Least Favorite, |
|---|---|---|
| | 5-Like Very Much) | 4-Most Favorite) |
| Water | Rating/St. Dev | Ranking/St. Dev |
| 0% Sucralose | 2.59/1.22 | 1.90/1.14 |
| 0.005% Sucralose | 3.04/1.13 | 2.71/0.82 |
| 0.01% Sucralose | 2.78/1.15 | 2.74/0.96 |
| 0.02% Sucralose | 2.59/1.15 | 2.65/1.33 |

TABLE 5

| | (1-Dislike, | (1-Least Favorite, |
|---|---|---|
| | 5-Like Very Much) | 4-Most Favorite) |
| 0.4% NaCl | Rating/St. Dev | Ranking/St. Dev |
| 0% Sucralose | 2.35/0.92 | 2.00/1.15 |
| 0.01% Sucralose | 2.86/0.95 | 2.70/1.05 |
| 0.02% Sucralose | 3.05/1.10 | 2.89/0.88 |
| 0.04% Sucralose | 2.78/1.20 | 2.41/1.21 |

TABLE 6

| | (1-Dislike, | (1-Least Favorite, |
|---|---|---|
| | 5-Like Very Much) | 4-Most Favorite) |
| 2% NaCl | Rating/St. Dev | Ranking/St. Dev |
| 0% Sucralose | 1.71/0.90 | 2.06/1.18 |
| 0.025% Sucralose | 2.06/0.96 | 2.74/0.86 |
| 0.05% Sucralose | 2.26/1.18 | 2.77/1.02 |
| 0.10% Sucralose | 2.23/1.26 | 2.42/1.29 |

Table 4 establishes that a relatively low level of Sucralose, 0.005%, is preferred in water alone. However, as increasing amounts of salt are added in Tables 5 and 6, the amount of Sucralose preferred increases, with 0.02% being preferred in the 0.4% NaCI solution and with 0.05% being preferred in the 2% NaCI solution. Thus, salty liquids having an equivalent saltiness to 0.02% NaCI in water, such as PEG based bowel cleansers, become most palatable with 0.02% Sucralose. Similarly, salty liquids having an equivalent saltiness to 2% NaCI in water, such as 60 mL of the phosphate salt bowel cleanser from Example 2 in 355 mL of water, become most palatable with 0.05% Sucralose.

FIG. 1 plots the increasing salinity of the solutions from Tables 4, 5, and 6 against the preferred concentration of Sucralose required to reduce the saltiness and increase the palatability of each solution. The graph establishes that as saltiness increases, the amount of sweetener that should be added to improve palatability linearly increases (R²=0.997). The X-axis NaCI and the Y-axis Sucralose concentrations represented by the correlation line from FIG. 1 are presented below in Table 7.

TABLE 7

| NaCl Concentration | Preferred Sucralose |
|---|---|
| (wt %) | Concentration (wt %) |
| 0.0 | 0.011 |
| 0.2 | 0.015 |
| 0.4 | 0.019 |
| 0.6 | 0.023 |
| 0.8 | 0.027 |
| 1.0 | 0.031 |
| 1.2 | 0.035 |
| 1.4 | 0.038 |
| 1.6 | 0.042 |
| 1.8 | 0.046 |
| 2.0 | 0.050 |
| 2.2 | 0.054 |
| 2.4 | 0.058 |
| 2.6 | 0.062 |
| 2.8 | 0.066 |
| 3.0 | 0.070 |

By determining the saltiness of any bowel cleanser in relation to NaCI/water solutions, the preferred concentration of Sucralose required to reduce the saltiness and increase the palatability of the solution may be determined from FIG. 1 and/or Table 7. Similarly, by extrapolating a Sucralose concentration value from FIG. 1 and/or Table 7 using an equivalent sweetness value from Example 4, the preferred amount of Ace-K, Saccharin, or a 5:1 Ace-K/Sucralose blend to add to the salty solution may be similarly determined.

### Example 10

To confirm the correlation of FIG. 1 in a bowel cleanser with multiple sweeteners, approximately 35 testers were asked to select the most palatable solution from five possibilities. Each of the five solutions included 60 mL of the phosphate salt bowel cleanser from Example 2 in 355 mL of water. This solution has an equivalent saltiness to the 2% NaCI/water solution of Example 9. The first of the five solutions lacked any sweetener, while an increasing concentration of sweetener was added to the remaining four. Tests were performed using Sucralose, the 5:1 Ace-K/Sucralose blend, Ace-K, or Saccharin as the sweetener.

Each of about thirty-five test subjects were asked to rate each of the five solutions on a scale of 1 to 4 with 4 being highly preferred and 1 being least preferred. Tables 8, 9, 10, and 11, below, present the average test data for each sweetener, respectively.

TABLE 8

| | Sucralose % (w/w) | | | | |
|---|---|---|---|---|---|
| | None | 0.0125 | 0.025 | 0.05 | 0.1 |
| Average | 2.13 | 2.56 | 2.72 | 3.22 | 2.88 |
| Standard Deviation | 1.13 | 1.05 | 1.05 | 1.21 | 1.24 |

TABLE 9

| | 5:1 Ace-K/Sucralose Blend % (w/w) | | | | |
|---|---|---|---|---|---|
| | None | 0.0125 | 0.025 | 0.05 | 0.1 |
| Average | 1.86 | 2.46 | 2.97 | 3.03 | 2.77 |
| Standard Deviation | 1.00 | 1.01 | 0.95 | 1.01 | 1.19 |

TABLE 10

| | Ace-K % (w/w) | | | | |
|---|---|---|---|---|---|
| | None | 0.025 | 0.05 | 0.1 | 0.2 |
| Average | 1.78 | 1.88 | 2.63 | 2.16 | 2.16 |
| Standard Deviation | 0.97 | 0.71 | 1.13 | 0.88 | 0.95 |

TABLE 11

| | Saccharin % (w/w) | | | | |
|---|---|---|---|---|---|
| | None | 0.0208 | 0.0416 | 0.0832 | 0.1664 |
| Average | 1.74 | 2.29 | 2.47 | 2.47 | 1.97 |
| Standard Deviation | 0.86 | 0.91 | 1.05 | 1.05 | 1.06 |

The data from the tables establish that in the phosphate salt bowel cleanser, 0.05% of the Sucralose, Ace-K/Sucralose blend, or Ace-K is preferred to decrease saltiness, while for Saccharin about 0.0832% may be preferred. Thus, FIG. 1 and/or Table 7 allow one to select a preferable concentration of sweetener to add to the bowel cleansing solution.

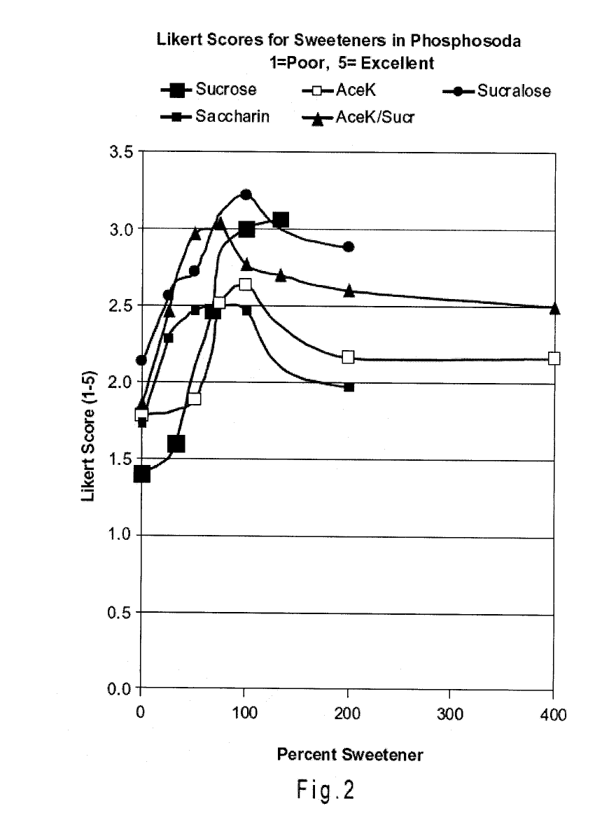

FIG. 2 plots the Likert preferability scores for each sweetener concentration on a scale of 1 to 5 with 5 being the most preferred. The sweetener concentration for each data point corresponds to those in Tables 8 through 11, above, with higher values on the X-axis corresponding to higher sweetener concentrations. FIG. 2 also shows the preference towards sucrose at concentrations of 5, 10, 15, and 20%.

The graph establishes that Sucralose is most preferred, even over sucrose in the bowel cleanser. Unlike in Example 5, where sucrose was slightly more preferred than Sucralose, in this study Sucralose was more preferred. Surprisingly, in bowel cleansers where sucrose rapidly degrades, the artificial sweetener Sucralose demonstrates an equivalent or even superior preference. The Ace-K/Sucralose blend was significantly superior to saccharin or Ace-K alone, but was not as preferred as Sucralose. Thus, while Sucralose alone was more preferred, the Sucralose/Ace-K sweetening system provides an alternative.

The inferior performance of Saccharin in this aspect is believed attributable to a bitter taste detected at higher concentrations. The equivalent preference values for the 0.0416 and 0.0832% saccharin solutions may be attributed to the fact that some tasters have a stronger negative reaction to this bitter taste, thus preferring the lower 0.0416% concentration. Thus, in addition to the test subjects preferring the sweetened phosphate salt bowel cleanser over the unsweetened liquid, a preferred level of sweetness when using a chlorinated sucrose isomer, such as Sucralose, was established.

### Example 11

To determine the preferred concentration of Sucralose to reduce the saltiness of a PEG based bowel cleanser, test subjects were asked to taste unsweetened and three sweetened solutions of NuLytely®. NuLytely® is an aqueous liquid including approximately 420 g of 3350 PEG, 5.72 g of sodium bicarbonate, 11.2 g of NaCI, and 1.48 g of KCI. It has a similar perceived saltiness to the similar GoLytely®, which is an aqueous liquid including approximately 236 g of 3350 PEG, 22.74 g of sodium sulfate, 6.74 g of sodium bicarbonate, 5.86 g of NaCI, and 2.97 g of KCI. Either solution has a saltiness that approximates the 0.4% NaCl/water solution of Example 9.

Each of thirty test subjects was asked to rate the four solutions on a scale of 1 to 5 with 5 being highly preferred and 1 being least preferred. The first solution included no sweetener. The second, third, and fourth solutions included 0.01%, 0.025%, or 0.05% (w/w) of Sucralose, respectively.

| | PEG Based Bowel Cleanser % Sucralose | | | |
|---|---|---|---|---|
| Test Subject | None | 0.01 | 0.025 | 0.05 |
| 1 | 1 | 3 | 5 | 3 |
| 2 | 2 | 3 | 2 | 2 |
| 3 | 2 | 2 | 4 | 4 |
| 4 | 3 | 4 | 2 | 1 |
| 5 | 2 | 2 | 3 | 2 |
| 6 | 2 | 4 | 3 | 2 |
| 7 | 2 | 4 | 4 | 1 |
| 8 | 3 | 4 | 4 | 2 |
| 9 | 1 | 2 | 2 | 1 |
| 10 | 2 | 3 | 2 | 1 |
| 11 | 1 | 4 | 3 | 2 |
| 12 | 2 | 2 | 1 | 1 |
| 13 | 1 | 2 | 4 | 5 |
| 14 | 1 | 3 | 2 | 2 |
| 15 | 3 | 4 | 1 | 1 |
| 16 | 1 | 2 | 3 | 4 |
| 17 | 3 | 3 | 5 | 2 |
| 18 | 2 | 3 | 2 | 1 |
| 19 | 2 | 2 | 2 | 1 |
| 20 | 3 | 2 | 4 | 4 |
| 21 | 2 | 2 | 3 | 4 |
| 22 | 1 | 2 | 2 | 3 |
| 23 | 2 | 2 | 2 | 2 |
| 24 | 4 | 3 | 2 | 2 |
| 25 | 2 | 3 | 4 | 4 |
| 26 | 1 | 3 | 4 | 4 |
| 27 | 3 | 3 | 2 | 2 |
| 28 | 2 | 4 | 4 | 5 |
| 29 | 2 | 2 | 2 | 2 |
| 30 | 1 | 2 | 4 | 4 |
| Average | 1.97 | 2.80 | 2.90 | 2.47 |
| Standard Deviation | 0.81 | 0.81 | 1.12 | 1.31 |

The data establishes that test subjects preferred the sweetened PEG based bowel cleanser by approximately 37% when compared with the unsweetened PEG cleanser. Thus, the subjects found sweetened PEG based bowel cleanser solutions significantly more palatable. Furthermore, the subjects preferred the PEG based bowel cleanser including 0.025% Sucralose in relation to the lower 0.01% Sucralose solution and substantially preferred the 0.025% solution over the much sweeter 0.05% Sucralose solution. Thus, in addition to the test subjects preferring the sweetened PEG based bowel cleanser over the unsweetened liquid, a preferred level of sweetness when using a chlorinated sucrose isomer, such as Sucralose, was established. Additionally, FIG. 1 and/or Table 7 in combination with Example 4 allow one to select a preferable concentration of sweetener to add to the PEG based bowel cleanser.

### Example 12

A further study was undertaken to determine which flavorings were preferred by test subjects to improve the palatability of FLEET® PHOSPHO-SODA® and to determine which flavorings were stable in FLEET® PHOSPHO-SODA®. The flavorings tested included sour apple, apple, banana, kiwi melon banana, mixed berry, berry, cantaloupe, caramel, celery, creamy cherry, cherry, wild cherry, chocolate chip cookie, chocolate wafer cookie, chocolate, German fudge brownie, mocha, chocolate fudge, citrus, citrus berry, coffee, cranberry, creme soda, cucumber, fruit punch, mixed fruit, herbal ginger, ginger, ginger ale, white grape, grape, tang grapefruit, lulo grapefruit, cran grapefruit, grapefruit, guanabana, guava, key lime, lime, margarita, lime, tequila lime, citrus blend margarita, mango melon, mangosteen, orange cream, orange, orange carrot, lemon orange carambola, passion fruit, peach, tropical peach, peach mango, pear, peppermint, pineapple - white sapote, pineapple, pitahaya, plum mulberry, raspberry, blue raspberry, raspberry, root beer, starfruit, strawberry, strawberry melon, sweet, tomato, tropical, tropical passion fruit, French vanilla cappuccino, vanilla, French vanilla, vanilla cookie, creamy vanilla, vanilla, raspberry vanilla, watermelon honeydew, wacky watermelon, watermelon, and yuzu.

Of these flavorings, apple, banana, kiwi melon banana, mixed berry, cherry, double fudge brownie, citrus, cantaloupe, fruit punch, mixed fruit, ginger ale, grape, grapefruit, citrus blend margarita, mango melon, mangosteen, plum mulberry, raspberry, root beer, strawberry melon, sweet, tomato, tropical, tropical passionfruit, and watermelon honeydew were selected on the basis of their ability to improve the palatability of the FLEET® PHOSPHO-SODA® when used in combination with the sweetener. These flavorings were then tested for stability in FLEET® PHOSPHO-SODA® over a three month period. Of these flavorings, Ginger Ale FAET253, Mangosteen FAES387, and Cola FAES389 were found to have acceptable stability in the phosphate salt bowel cleanser. Thus, Ginger Ale FAET253, Mangosteen FAES387, and Cola FAES389 are the preferably flavorings to improve the palatability of FLEET® PHOSPHO-SODA® when used in combination with the sweetener.

All percentages, ratios, parts, and other amounts described herein, unless otherwise noted, are weight/weight percentages, ratios, parts and amounts. All flavorings were obtained from WILD Flavors, Inc., of Erlanger, Ky. While various embodiments of the invention have been described, it will be apparent to those of ordinary skill in the art that other embodiments and implementations are possible within the scope of the invention. Accordingly, the invention is not to be restricted except in light of the attached claims and their equivalents.

1. A composition for bowel cleansing comprising:
from 0.01 to 0.1% of a sweetener selected from the group consisting of a chlorinated sucrose isomer, acesulfame potassium, saccharin, and mixtures thereof; and
a bowel cleanser having a perceived saltiness equivalent to from 0.2 to 2.6% sodium chloride in water.

2. The composition of claim 1, where the chlorinated sucrose isomer is Sucralose.

3. The composition of claim 1, where the sweetener is a mixture of chlorinated sucrose isomer and acesulfame potassium in a 1 to from 4 to 6 ratio by weight, respectively.

4. The composition of claim 2, comprising from 0.04 to 0.09% of the sweetener and where the perceived saltiness of the bowel cleanser is equivalent to from 1.8 to 2.2% sodium chloride in water.

5. The composition of claim 4, comprising from 0.04 to 0.06% of the sweetener, where the sweetener is selected from the group consisting of the Sucralose isomer, the acesulfame potassium, and mixtures thereof.

6. The composition of claim 2, comprising from 0.01 to 0.04% of the sweetener and where the perceived saltiness of the bowel cleanser is equivalent to from 0.2 to 0.6% sodium chloride in water.

7. The composition of claim 6, comprising from 0.01 to 0.03% of the sweetener, where the sweetener is selected from the group consisting of the Sucralose isomer, the acesulfame potassium, and mixtures thereof.

8. The composition of claim 1, further comprising a flavoring selected from the group consisting of mangosteen, cola, ginger ale, and combinations thereof.

9. The composition of claim 8, including from 0.3 to 2.3% of the flavoring.

10. The composition of claim 1, where the bowel cleanser comprises at least one active ingredient selected from the group consisting of a phosphate salt and polyethylene glycol.

11. The composition of claim 1, where the bowel cleanser comprises a phosphate salt selected from the group consisting of monobasic sodium phosphate, dibasic sodium phosphate, and combinations thereof.

12. The composition of claim 11, where the phosphate salt has a pH from 4.4 to 5.2 in water.

13. The composition of claim 11, where the phosphate salt comprises water and from 0.05 to 1.5 gram/mL of monobasic sodium phosphate and from 0.02 to 0.6 gram/mL of dibasic sodium phosphate.

14. The composition of claim (1), where the bowel cleanser comprises a polyethylene glycol and from 0.4 to 1% salt.

15. A composition for bowel cleansing comprising a Sucralose concentration and a perceived saltiness equivalent to a sodium chloride in water concentration corresponding to the line of FIG. 1.

16. A method for reducing saltiness of an orally consumed substance having a perceived saltiness equivalent to from 0.2 to 2.6% sodium chloride in water, comprising:
combining the orally consumed substance with from 0.01 to 0.1% of a sweetener selected from the group consisting of a chlorinated sucrose isomer, acesulfame potassium, saccharin, and mixtures thereof.

17. The method of claim 16, where the orally consumed substance comprises a bowel cleanser.

18. The method of claim 17, where the bowel cleanser comprises at least one active ingredient selected from the group consisting of a phosphate salt and polyethylene glycol.

19. The method of claim 16, where the orally consumed substance comprises a phosphate salt selected from the group consisting of monobasic sodium phosphate, dibasic sodium phosphate, and combinations thereof.

20.-25. (canceled)

26. A method for improving the palatability of a salty laxative, comprising:
combining a concentration having a perceived saltiness equivalent to from 0.2 to 2.6% sodium chloride in water of the laxative with from 0.01 to 0.07% of a sweetener including Sucralose.

27.-30. (canceled)
